(19) United States
(12) Patent Application Publication
(10) Pub. No.: US 2025/0143708 A1
(43) Pub. Date: May 8, 2025

(54) HYBRID SHUNT SYSTEM FOR CAROTID ENDARTERECTOMY

(71) Applicant: THE CATHOLIC UNIVERSITY OF KOREA INDUSTRY-ACADEMIC COOPERATION FOUNDATION, Seoul (KR)

(72) Inventors: Jai Ho CHOI, Seoul (KR); Woo Cheul CHO, Anyang (KR)

(21) Appl. No.: 18/930,574

(22) Filed: Oct. 29, 2024

(30) Foreign Application Priority Data
Nov. 8, 2023 (KR) 10-2023-0153850

(51) Int. Cl. A61B 17/12; A61B 17/00

(52) U.S. Cl. CPC A61B 17/12109; A61B 17/12045; A61B 17/12136; A61B 2017/00477; A61B 2017/1205

(57) ABSTRACT

A hybrid shunt system for carotid endarterectomy.

### CROSS-REFERENCE TO RELATED APPLICATION

This application claims priority from Korean Patent Application No. 10-2023-0153850, filed on Nov. 8, 2023, which is hereby incorporated by reference for all purposes as if fully set forth herein.

### BACKGROUND

### Field

The disclosure relates to a hybrid shunt system for carotid endarterectomy (sometimes abbreviated as “hybrid shunt system”), which allows for a smaller incision, a much easier procedure, a shorter procedure time, and the rapid and safe insertion of an occlusion balloon into a precise location within the vessel via a guide wire, even when the vessel is obscured by the neck bone.

### Description of Related Art

The carotid artery may be divided into an internal carotid artery (ICA) facing into the skull, an external carotid artery (ECA) facing outside the skull, and a common carotid artery (CCA) in which the internal and external carotid arteries are combined.

Carotid stenosis is usually caused by cholesterol depositing in blood vessels at or near the junction of the outer and inner carotid arteries, and surgical treatments such as carotid artery stenting (CAS) or carotid endarterectomy (CEA) or drugs using antiplatelet drugs and anticoagulants are known.

The differences between carotid endarterectomy (CEA) and carotid artery stenting (CAS) may be summarized as shown in Table 1.

TABLE 1

| | | CEA | CAS |
|---|---|---|---|
| Surgery type | | direct surgical | widen legion with pressure |
| | | removal of plaque | by interventional surgery |
| Procedure time | | 3-4 hr | −1 hr |
| Procedure | (domestic) | 2 | 8 |
| ratio | (overseas) | 8 | 2 |

| | | |
|---|---|---|
| Clinical evaluation | superiority | non-inferiority |

The carotid endarterectomy according to the disclosure may be performed using a carotid shunt or using a clamp and a catheter. Among them, the procedure using Pruitt F3® carotid shunt (LeMaitre) may cause bleeding because an integral carotid shunt is inserted into the incised blood vessel, and especially in the case of high bifurcation, pierce the blood vessel as the carotid shunt must be inserted into the blood vessel without being visible. Further, it is not imported due to its low domestic use.

### SUMMARY OF THE INVENTION

The disclosure aims to provide a hybrid shunt system for carotid endarterectomy which allows for a smaller incision, a much easier procedure, a shorter procedure time, and the rapid and safe insertion of an occlusion balloon into a precise location within the vessel via a guide wire, even when the vessel is obscured by the neck bone, as compared with integral carotid artery shunts, while addressing the conventional issues described above.

A hybrid shunt system for carotid endarterectomy proposed in the disclosure may comprise an internal carotid artery inserter having an internal carotid artery occlusion balloon contracting/expanding by entry and exit of a working fluid, such as a gas or saline, and provided at one end of a tube, and a hub having a blood flow opening/closing port with a blood flow opening/closing cock and a working fluid entry/exit port at another end of the tube, a common carotid artery inserter having an occlusion balloon at one end of the tube and a hub having a blood flow opening/closing port with a blood flow opening/closing cock and a working fluid entry/exit port, and a connector bypassing a blood flow during a carotid endarterectomy (CEA) procedure by connecting the blood flow opening/closing port of the common carotid artery inserter and the blood flow opening/closing port of the internal carotid artery inserter.

Further, in embodiments of the disclosure, the hybrid shunt system may have a structure that may be separated into two or three parts.

Further, in embodiments of the disclosure, the connector may be a three-way cock.

Since the hybrid shunt system according to embodiments of the disclosure is configured so that the internal carotid artery inserter and the common carotid artery inserter are disassembled and assembled, it provides the advantage of rapid, safe, and accurate insertion of an occlusion balloon into the intervened vessel with a 5 to 10 millimeter reduction as compared to using an integral shunt such as the Pruitt F3® carotid shunt, as well as much convenience and a shortened procedure time.

Further, even if the blood vessel is covered by the neck bone and is not visible, an occluded balloon may be inserted at an accurate location in the blood vessel through a guide wire.

Other features of the disclosure will be more apparent from the following description taken in conjunction with the accompanying drawings.

### BRIEF DESCRIPTION OF DRAWINGS

The above and other aspects, features, and advantages of the disclosure will be more clearly understood from the following detailed description, taken in conjunction with the accompanying drawings, in which:

FIG. 1 is a view illustrating a first embodiment of the disclosure;

FIG. 2 is a view illustrating a second embodiment of the disclosure; and

FIGS. 3A to 3J are views illustrating a procedure of a hybrid shunt system according to the disclosure in chronological order.

### DESCRIPTION OF THE PREFERRED EMBODIMENTS

Hereinafter, some embodiments of the disclosure are described in detail with reference to the accompanying drawings, and detailed descriptions of known configurations and functions that may obscure the gist of the disclosure are omitted.

In the accompanying drawings, the same reference numerals are assigned to the same components, although they are shown in different drawings.

In the disclosure, the singular forms “a,” “an,” and “the” are intended to include the plural forms as well, unless the context clearly indicates otherwise.

When an element comprises, has, or includes parts, the element may include other parts unless the element only comprises, has, or includes the parts.

The terms “first,” “second,” “A,” “B,” “(a),” and “(b)” in the disclosure are used merely to distinguish an element from another, and the essence, order, sequence, or number of the elements should not be limited thereby.

When an element is referred to as being “connected” or “coupled” to another element, it can be directly connected or coupled to the other element or intervening elements may be present.

When an element is referred to as being “after,” “subsequent to,” “next to,” or “before” another element, the element may be not consecutive to the other element unless the element is “directly” or “immediately” after, subsequent to, next to, or before the other element.

Further, when a numerical value of a component or corresponding information (e.g., a level, etc.) thereof is mentioned, the numerical value or corresponding information may be interpreted as including an error range generated by various factors (e.g., a process factor, an internal/external impact, noise, etc.) even without an explicit description.

### EMBODIMENTS

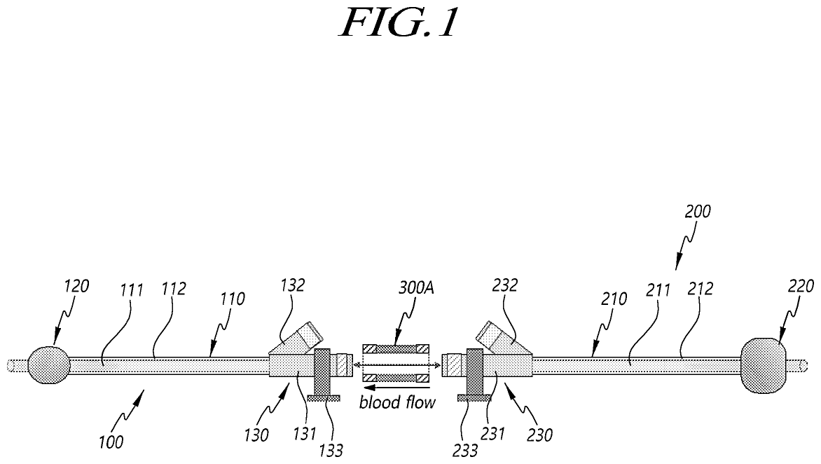

FIG. 1 is a view schematically illustrating a first embodiment of the disclosure, and may include an internal carotid artery inserter 100, a common carotid artery inserter 200, and a connector 300A.

The internal carotid artery inserter 100 may include a tube 110, an internal carotid artery occlusion balloon 120 provided at one end of the tube 110, and a hub 130 provided at the other end of the tube 110.

The tube 110 may have, e.g., a concentric structure in which a second flow path 112 surrounds a first flow path 111. Further, the first flow path 111 is longer than the second flow path 112. During a CEA procedure, blood bypasses the first flow path 111, and a working fluid such as saline enters/exits or fills the second flow path 112.

The internal carotid artery occlusion balloon 120 may be formed of a material such as polyurethane, silicone, or latex so as to be in the form of a balloon having a diameter of 5.0 mm and a length of 10 mm during maximum expansion.

The hub 130 includes a blood flow opening/closing port 131 where the blood flow opening/closing cock 133 is installed and a working fluid entry/exit port 132 having one side communicating with the second flow path 112 of the tube 110.

The tube 110 is coupled to one end of the blood flow opening/closing port 131, and the connector 300A is connected to the other end.

The blood flow opening/closing port 131 and the connector 300A may be connected, e.g., in a snap-fit manner. Further, a working fluid supply means (not shown) may be connected to the other side of the working fluid entry/exit port 132 in a snap-fit manner.

Like the internal carotid artery inserter 100, the common carotid artery inserter 200 may include a tube 210, a common carotid artery occlusion balloon 220 provided at one end of the tube 210, and a hub 230 provided at the other end of the tube 210.

The tube 210 of the common carotid artery inserter 200 may also be a multi-lumen tube in which the first flow path 211 is surrounded by the second flow path 212. Similarly, during the CEA procedure, blood bypasses the first flow path 211, and the working fluid enters and exits the second flow path 212.

The common carotid artery occlusion balloon 220 may be formed of a material such as polyurethane, silicone, or latex so as to be in the form of a balloon having a diameter and a length of 10 mm during maximum expansion.

Like the hub 130 of the internal carotid artery inserter 100, the hub 230 of the common carotid artery inserter 200 includes a blood flow opening/closing port 231 and a working fluid entry/exit port 232, and a blood flow opening/closing cock 233 is installed on the path of the blood flow opening/closing port 231.

The connector 300A is in the form of a tube connecting the blood flow opening/closing port 131 of the internal carotid artery inserter 100 and the blood flow opening/closing port 231 of the common carotid artery inserter 200, and forms a bypass that allows blood to be supplied to the brain during a CEA procedure. The central portion of the connector 300A may have a bellows structure (wrinkle tube shape) that may be bent or unbent.

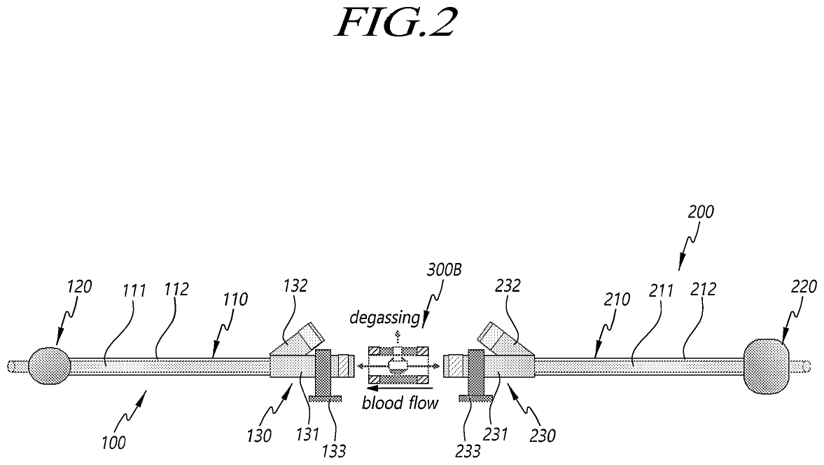

Next, a hybrid shunt system of a second embodiment is described with reference to FIG. 2. In the same manner as in the first embodiment, the hybrid shunt system of the second embodiment may include an internal carotid artery inserter 100, a common carotid artery inserter 200, and a connector 300B.

The internal carotid artery inserter 100 is provided with an internal carotid artery occlusion balloon 120 at one end of the tube 110, and a hub 130 at the other end.

The tube 110 is a multi-lumen tube including a first flow path 111 through which blood bypasses and a second flow path 112 where a working fluid is charged, and the first flow path 111 and the second flow path 112 may be concentrically arranged.

The internal carotid artery occlusion balloon 120 may be formed of a flexible material such as polyurethane or silicone so as to be in the form of a balloon having a diameter of 5.0 mm and a length of 10 mm during maximum expansion.

The hub 130 of the internal carotid artery inserter 100 includes a blood flow opening/closing port 131 and a working fluid entry/exit port 132, and a blood flow opening/closing cock 133 is installed on the path of the blood flow opening/closing port 131.

In the same manner as in the first embodiment described above, the common carotid artery inserter 200 may include a tube 210 having a double channel structure in which the first flow path 211 and the second flow path 212 are concentrically arranged, a common carotid artery occlusion balloon 220 provided at one end of the tube 210, and a hub 230 mounted at the other end of the tube 210.

The common carotid artery occlusion balloon 220 may be formed of a material such as polyurethane, silicone, or latex so as to be in the form of a balloon having a diameter and a length of 10 mm during maximum expansion.

In the same manner as in the first embodiment, the hub 230 of the common carotid artery inserter 200 has a blood flow opening/closing port 231 and a working fluid entry/exit port 232, and the blood flow opening/closing cock 233 is installed on the path of the blood flow opening/closing port 231.

Meanwhile, unlike the connector 300A of the first embodiment, the connector 300B of the second embodiment may be a three-way cock. An example of the three-way cock is a T-type three-way ball valve, and one of the three openings formed in the connector 300B is used for degassing.

As described above, the internal carotid insert 100 and the common carotid insert 200 of the first embodiment and the second embodiment have the same configuration, but the connectors 300A and 300B have different configurations.

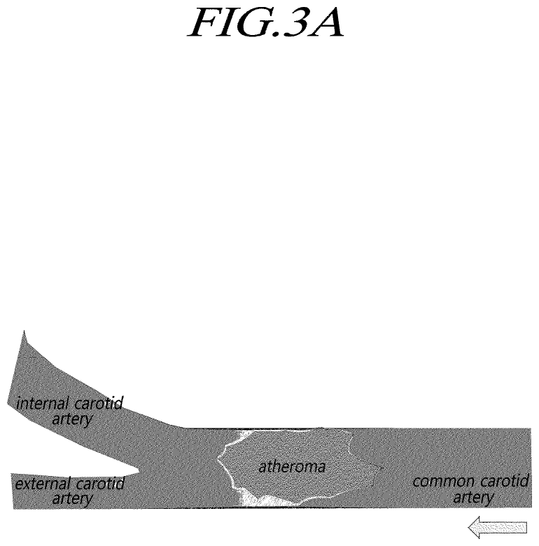

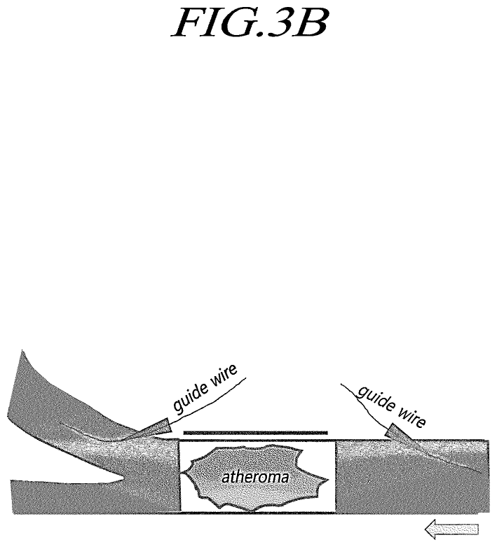

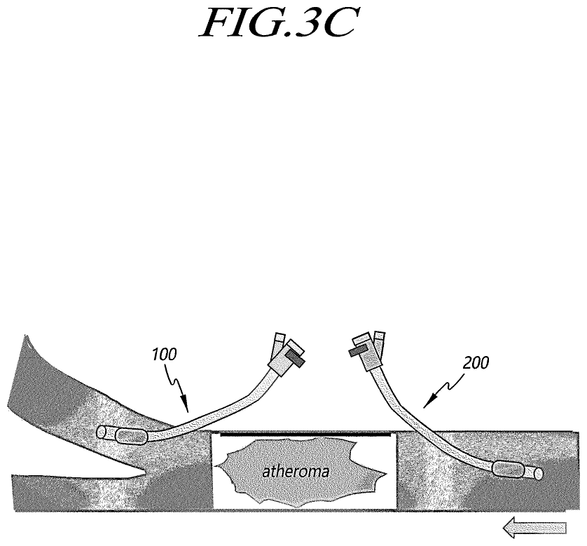

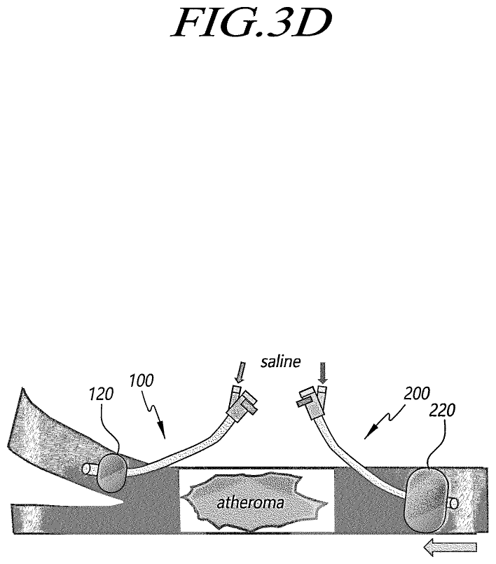

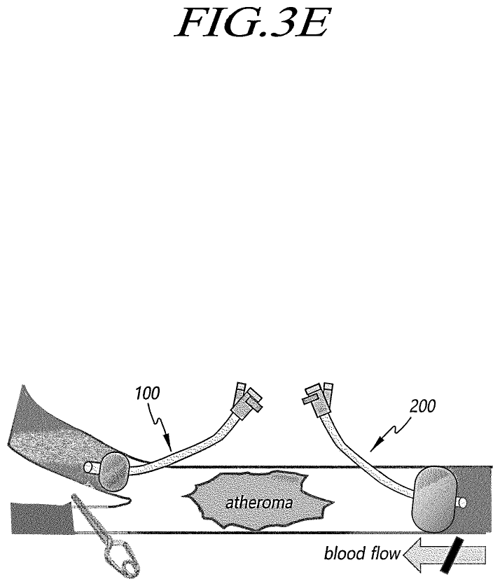

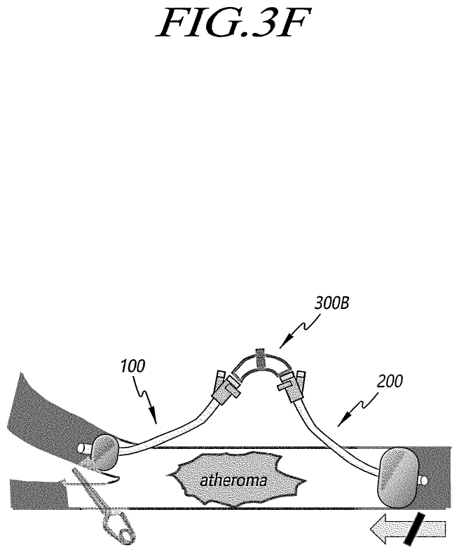

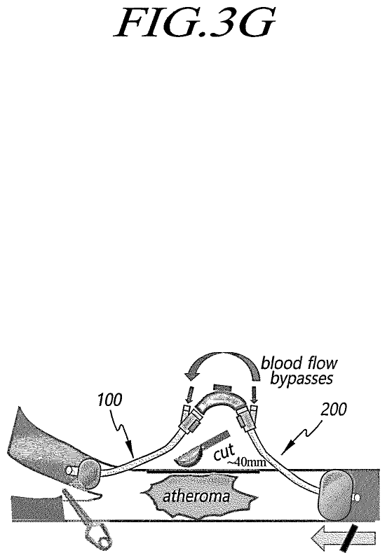

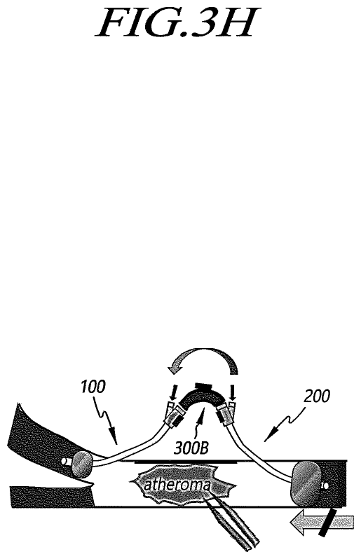

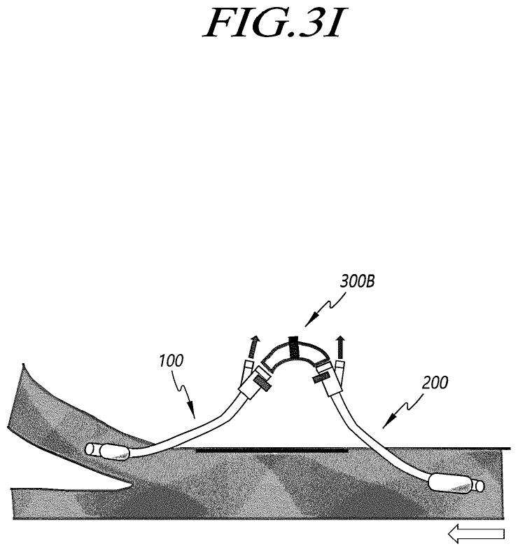

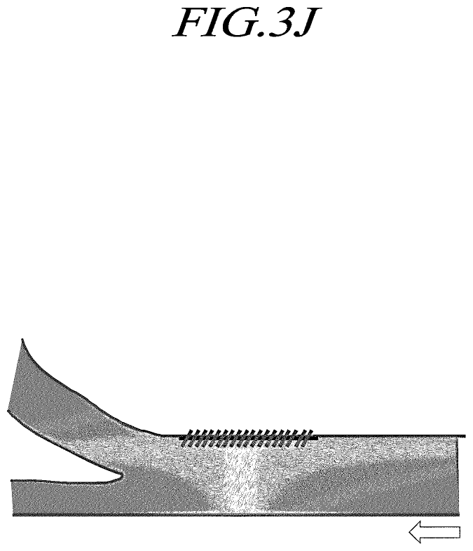

Next, a procedure of the hybrid shunt system according to the disclosure is described. In the procedure of FIGS. 3A to 3J, the hybrid shunt system of the second embodiment is used, but the same is true even when the hybrid shunt system of the first embodiment is used.

First, the neck skin of the area where the atheroma occurred is incised to expose the carotid artery (FIG. 3A), and then the guide wire is inserted between the blood vessels above and below the lesion (FIG. 3B).

Thereafter, the internal carotid artery occlusion balloon 120 is inserted through the guide wire inserted into the internal carotid artery, saline solution is injected into the internal carotid artery occlusion balloon 120 to expand the balloon, and the guide wire is removed from the blood vessels (FIGS. 3C and 3D). Similarly, the common carotid artery occlusion balloon 220 is inserted through the guide wire inserted into the common carotid artery, saline solution is injected into the common carotid artery occlusion balloon 220 to expand the balloon, and the guide wire is removed from the blood vessels (FIGS. 3C and 3D). Accordingly, the internal carotid artery occlusion balloon 120 and the common carotid artery occlusion balloon 220 are installed at the correct positions in the blood vessel, and blood flow is blocked.

Then, the external carotid artery is tightly bit with a clamp to block blood flow (FIG. 3E), and the blood flow opening and closing ports of the internal carotid artery inserter 100 and the common carotid artery inserter 200 are connected to two opposite ends, respectively, of the connector 300B (FIG. 3F).

Then, the blood flow opening/closing cock of the internal carotid artery inserter 100 and the blood flow opening/closing cock of the common carotid artery inserter 200 are operated in an open state to bypass the blood flow, the lesion area is cut to a predetermined length (e.g., a length of 40 mm or less) (FIG. 3G). In this process, the valve of the connector 300B is operated to degass (discharge a trace amount of blood suspected of air contact).

Thereafter, the atheroma is removed (FIG. 3H), saline solution injected into the internal carotid artery occlusion balloon 120 and the common carotid artery occlusion balloon 220 is discharged to contract the balloon, and the blood flow opening/closing cocks are closed to separate the hybrid shunt system from the patient (FIG. 3I).

Finally, the blood vessels are sutured, and the incised skin is sutured (FIG. 3j).

While some embodiments of the disclosure have been described in detail, these are merely examples. It will be appreciated by one of ordinary skill in the art that various changes/modifications may be made without departing from the technical spirit of the claims.

As a modified example, in the hybrid shunt system of the disclosure, the connector may be integrated with any one of the blood flow opening/closing port of the internal carotid artery inserter or the blood flow opening/closing port of the common carotid artery inserter. Further, the maximum expansion diameter of the internal carotid artery occlusion balloon may be 4 to 8 mm, the maximum expansion diameter and length of the common carotid artery occlusion balloon may be 8 to 14 mm, and the diameter of the first flow path bypassed by blood flow may be 3 to 5 mm (9 to 15 Fr). Further, the total length in a state in which the internal carotid artery inserter, the connector, and the common carotid artery inserter are assembled may be 10 to 15 cm.

Therefore, the protection scope and technical configuration of the disclosure are not limited to the above-described first embodiment and the above-described second embodiment, and should be interpreted to include at least the following matters.

[1] A hybrid shunt system for carotid endarterectomy comprising an internal carotid artery inserter having an internal carotid artery occlusion balloon provided at one end of a tube and a hub having a blood flow opening/closing port with a blood flow opening/closing cock and a working fluid entry/exit port at another end of the tube, a common carotid artery inserter having a common carotid artery occlusion balloon at one end of the tube and a hub having a blood flow opening/closing port with a blood flow opening/closing cock and a working fluid entry/exit port at another end of the tube, and a connector connecting the blood flow opening/closing ports of the common carotid artery inserter and the internal carotid artery inserter.

[2] In [1] described above, the hybrid shunt system for carotid endarterectomy in which the connector is a three-way cock.

[3] In [1] or [2] described above, the hybrid shunt system for carotid endarterectomy in which the tube is a multi-lumen tube including a first flow path through which blood may flow and a second flow path through which the working fluid may flow, and has a concentric structure where the first flow path passes inside the second flow path.

[4] In [1] to [3] described above, the hybrid shunt system for carotid endarterectomy in which one end of the first flow path passes through the occlusion balloon and one end of the second flow path communicates with the occlusion balloon.

[5] In [1] to [4] described above, the hybrid shunt system for carotid endarterectomy in which the connector is integrally formed with any one of the blood flow opening/closing port of the internal carotid artery inserter or the blood flow opening/closing port of the common carotid artery inserter.

What is claimed is:

1. A hybrid shunt system for carotid endarterectomy comprising:
an internal carotid artery inserter having an internal carotid artery occlusion balloon contracting/expanding with entry and exit of a working fluid and provided at one end of a tube, and a hub having a blood flow opening/closing port with a blood flow opening/closing cock and a working fluid entry/exit port at another end of the tube;
a common carotid artery inserter having a common carotid artery occlusion balloon contracting/expanding with the entry and exit of the working fluid at one end of the tube, and a hub having a blood flow opening/closing port with a blood flow opening/closing cock and a working fluid entry/exit port at another end of the tube; and
a connector bypassing a blood flow during a carotid endarterectomy (CEA) procedure by connecting the blood flow opening/closing port of the common carotid artery inserter and the blood flow opening/closing port of the internal carotid artery inserter.

2. The hybrid shunt system of claim 1, wherein the connector is a three-way cock.

3. The hybrid shunt system of claim 2, wherein the tube is a multi-lumen tube including a first flow path through which blood may flow and a second flow path through which the working fluid may flow.

4. The hybrid shunt system of claim 3, wherein the tube has a concentric structure where the first flow path passes inside the second flow path.

5. The hybrid shunt system of claim 3, wherein one end of the first flow path passes through the occlusion balloon and one end of the second flow path communicates with the occlusion balloon.

6. The hybrid shunt system of claim 1, wherein the connector is integrally formed or assembled with any one of the blood flow opening/closing port of the internal carotid artery inserter or the blood flow opening/closing port of the common carotid artery inserter.
